(19) United States
(12) Patent Application Publication
(10) Pub. No.: US 2025/0087014 A1
(43) Pub. Date: Mar. 13, 2025

(54) METHOD AND SYSTEM FOR DETECTING A VITAL SIGN

(71) Applicant: TRINAMIX GMBH, Ludwigshafen (DE)

(72) Inventors: Muhammad Muneeb HASSAN, Ludwigshafen (DE); Christian LENNARTZ, Ludwigshafen (DE)

(21) Appl. No.: 18/729,047

(22) PCT Filed: Feb. 14, 2023
(86) PCT No.: PCT/EP23/53606

§ 371 Date: Jul. 15, 2024

(30) Foreign Application Priority Data
Feb. 15, 2022 (EP) 22156866.0
Feb. 25, 2022 (EP) 22158798.3

(51) Int. Cl. G06V 40/10; A61B 5/00; G06V 10/75; G06V 10/82

(52) U.S. Cl. CPC G06V 40/10; A61B 5/0059; A61B 5/7264; G06V 10/751; G06V 10/82; A61B 2576/00

(57) ABSTRACT

Disclosed herein is a method for detecting a vital sign, the method including the steps of a) generating or receiving an image data set representing at least one reflection image of a speckle pattern produced by coherent electromagnetic radiation reflected from an object, b) determining a speckle contrast of the speckle pattern, c) determining a vital sign measure based on the determined speckle contrast, and d) providing the vital sign measure.

### FIELD OF THE INVENTION

The invention relates to a method, to a system and to a computer program for detecting a vital sign indicative of a presence of a living subject. The invention particularly concerns the detection whether or not a living subject is presented to a camera, in particular, as part of an authentication process.

### BACKGROUND OF THE INVENTION

For gaining access to an electronic device, it is generally required to verify whether a user is actually allowed to do so. Verification can generally be achieved by entering the correct password or passcode into the device as part of an authentication process. Alternatively, a biometric authentication process may be used comprising fingerprint authentication employing a fingerprint sensor or facial recognition including matching a human face from a recorded digital image against one or more reference images.

With regard to facial recognition, a situation may arise in which closely related faces of different persons, e.g. siblings or twins cannot be distinguished anymore. As a consequence, an unauthorized person may gain access to an electronic device.

To avoid such a situation, it has been suggested to use additional authentication processes and only after having passed all of these authentication processes, access to an electronic device will be allowed.

For example, in U.S. Pat. No. 10,719,692 B2 it was suggested to enhance the security of a facial recognition authentication process by using subepidermal images of a user to assess subepidermal features such as blood vessels when a device is attempting to authenticate a user. The subepidermal features shall be compared to templates of subepidermal features for an authorized user of the device. In the suggested process, the user's face has to be captured with sufficient resolution in the image to allow image processing of the subepidermal features of the user's face.

In U.S. Pat. No. 9,971,948 B1, it was suggested to apply imaging of blood vessel patterns below the skin of a human subject including illuminating the skin with a serious of very short pulses of infrared radiation. The pulses are modulated by the blood in subcutaneous vessels and the surrounding tissue. Based on this modulation of the pulses, an image of the blood vessels located beneath the body surface shall be generated and used in the authentication process. To receive a meaningful result, it can generally be expected that the skin have to be illuminated for a period of at least one full heartbeat.

### SUMMARY OF THE INVENTION

The present invention is based on the objective of providing a method, a system and a computer program enabling a detection of a living subject presented to a camera. In particular, a method, a system and a computer program shall be provided enabling a detection of whether an object presented to a camera is actually a living subject. Preferably, the detection may be accomplished comparatively fast. It is further preferred that no expensive hardware is required for the detection.

According to the present invention, a method for detecting a vital sign is proposed that comprises the steps of
generating or receiving an image data set representing at least one reflection image of a speckle pattern produced by coherent electromagnetic radiation reflected from an object,
determining a speckle contrast of the speckle pattern,
determining a vital sign measure based on the determined speckle contrast, and
providing the vital sign measure.

The invention is based on the recognition that a moving body fluid or moving particles in the body of a living subject, such as blood cells, in particular, red blood cells, interstitial fluid, transcellular fluid, lymph, ions, proteins and nutrients, may cause a motion blur to reflected light while the remaining parts of the body are still and hence do not cause motion blur. Thus, if coherent electromagnetic radiation is reflected by moving scattering particles like red blood cells, the speckle pattern fluctuates and speckles become blurred. As a consequence of this blurring, the speckle contrast decreases. Therefore, the speckle pattern and the speckle contrast derived from the speckle pattern contain information about whether or not the illuminated object is a living subject. Speckle contrast values are generally distributed between 0 and 1. When illuminating an object, the value 1 may represent no motion and the value 0 may represent the fastest motion of particles thus causing the most prominent blurring of the speckles. Since the vital sign measure is determined based on the speckle contrast, the lower is the speckle contrast value, the higher is the certainty that a corresponding vital sign measure indicates the presence of a living subject. On the contrary, the higher is the speckle contrast value, the higher is the certainty that a corresponding vital sign measure indicates the presence of an object that is not a living subject.

Thereby, the method according to the invention allows detecting a vital sign in a reliable and efficient manner. The method according to the invention thus allows detecting whether an object presented to a camera is a living subject.

In particular, one single reflection image, only, is sufficient to obtain enough information for reliably detecting a vital sign. For recording the one single reflection image, only one single illumination of the object with coherent electromagnetic radiation is sufficient. With the method, a vital sign measure can thus be obtained comparatively fast.

Based on the vital sign measure, it is possible to reliably decide whether a living subject has been presented to a camera. It is a particular advantage, that the method is robust against spoofing, e.g., by presenting a mask or the like that replicates the authorized user's face to the camera.

It is a further advantage of the method according to the invention that the reflection image represented by the image data set can be recorded using standard equipment, e.g., a standard laser and a standard camera, e.g., comprising a charge coupled device (CCD) and/or a complementary metal oxide semiconductor (CMOS) sensor element.

In the framework of this specification, a vital sign is indicative of the presence of a living subject. In other words, a vital sign is any sign suitable for distinguishing a living subject from no-living material. Thus, an object of which a vital sign can be detected is considered a living subject. Herein, a vital sign particularly relates to the presence of a moving body fluid or moving particles in the body of a living subject. In this regard, the presence of blood flow, e.g. of red blood cells, is a preferred vital sign. However, other moving body fluids or moving particles present in the body of a living subject may also be used as a vital sign such as interstitial fluid, transcellular fluid, lymph, ions, proteins and nutrients. Preferably, the vital sign is detectable by analysing a speckle pattern, e.g., by detecting blurring of speckles caused by moving body fluid or moving particles in the body of a living subject. This blurring may decrease a speckle contrast in comparison to a case in which no moving body fluid or moving particles are present.

Coherent electromagnetic radiation refers to electromagnetic radiation that is able to exhibit interference effects. It may also include partial coherence, i.e. a non-perfect correlation between phase values. Preferably, the electromagnetic radiation has a wavelength in the wavelength range of 800 nm to 1000 nm, preferably, of 850 nm to 950 nm, even more preferably, of 930 nm to 950 nm, in particular, of 935 nm to 945 nm. It is thus preferred that electromagnetic radiation in the infrared wavelength range is used which has the advantage that the electromagnetic radiation may not be perceived visually by a user. This may be of particular advantage in an authentication process. On the one hand, the user may not recognize the way the authentication is performed. On the other hand, a user may be comparatively less distracted during the authentication process.

A speckle pattern is an interference pattern produced by coherent electromagnetic radiation reflected from an object, e.g., reflected from an outer surface of that object or reflected from an inner surface of that object. A speckle pattern typically occurs in diffuse reflections of coherent electromagnetic radiation such as laser light. Within the speckle pattern, the spatial intensity of the coherent electromagnetic radiation varies randomly due to interference of coherent wave fronts.

A speckle contrast may represent a measure for a mean contrast of an intensity distribution within an area of a speckle pattern. In particular, a speckle contrast K over an area of the speckle pattern may be expressed as a ratio of standard deviation a to the mean speckle intensity <I>, i.e.,

$$K = \frac{σ}{< I >}$$

Speckle contrast values are generally distributed between 0 and 1.

A vital sign measure, preferably, represents a measure indicative of whether the object from which the coherent electromagnetic radiation was reflected shows a vital sign. The vital sign measure is determined based on the speckle contrast. Thus, the vital sign measure may depend on the determined speckle contrast. If the speckle contrast changes, the vital sign measure derived from the speckle contrast may change accordingly. A vital sign measure may be a single number or value that may represent a likelihood that the object is a living subject.

Preferably, for determining the speckle contrast, the complete speckle pattern of the reflection image is used. Alternatively, for determining the speckle contrast, a section of the complete speckle pattern may be used. The section of the complete speckle pattern, preferably, represents a smaller area of the speckle pattern than an area of the complete speckle pattern. The section of the speckle pattern may be obtained by cropping the reflection image.

Preferably, the method comprises a step of authenticating an object, e.g., a user, based on the determined vital sign measure. Authenticating an object based on the determined vital sign measure has the advantage that it can be verified that actually a living being is presented to a camera and not a mask or the like used for gaining unauthorized access. Preferably, authenticating an object includes matching at least one biometric feature obtained from the object trying to gain access with at least one reference or template biometric feature associated with an authorised object. In case the at least one biometric feature obtained from the object matches with the at least one reference or template biometric feature associated with an authorised object, access may be approved. Biometric features may be, e.g., a feature of the face such as the relative position or size of the eyes, the mouth or the nose, a fingerprint, a hand geometry, a palm print, or an iris. For example, in an authentication process, in a first step, a biometric authentication can be carried out and if the first authentication step has been passed successfully, a second authentication step may be carried out that includes the determination of a vital sign measure to verify that actually a living being has been presented to a camera and not a spoofing mask or the like.

In the method, it is possible that the image data set represents at least two reflection images of speckle patterns produced by coherent electromagnetic radiation reflected from the object. Preferably, for each of at least two reflection images, an individual vital sign measure may be determined based on the speckle pattern of a respective one of the at least two reflection images. As a result, from the several reflection images each of them representing a speckle pattern, a number of vital sign measures may be determined, respectively. Afterwards, the determined individual vital sign measures may be provided, e.g., to a user or for further processing by the same or a different device, e.g., for conducting an authentication process.

The method may comprise that a map of a number of vital sign measures is generated. A vital sign measure map can be generated from a speckle contrast map. A speckle contrast map may comprise a number of speckle contrast values each being associated with a position, e.g. a position on the object at which a corresponding reflection image has been recorded. A speckle contrast map may be represented using a matrix having speckle contrast values as matrix entries.

A vital sign measure map may be represented by a matrix containing individual vital sign measures as matrix entries. A map of vital sign measures may represent a spatial distribution of the determined vital sign measure. Each of the vital sign measures may be associated with a different position on the object at which the object has been illuminated with coherent electromagnetic radiation for recording the corresponding speckle pattern. Thus, it can be beneficial, if a vital sign measure is associated with a position on the object. For generating the map of vital sign measures, the positions associated with the respective vital sign measures can be taken into account. Thus, a map may represent a spatial distribution of vital sign measures, e.g., in the coordinate system of the illuminated object. For example, a position associated with a vital sign measure may be represented by a spatial coordinate in the coordinate system of the illuminated object.

Accordingly, as part of the method, it may be preferred that at least two vital sign measures, each being associated with a different spatial position on the object are determined and provided as a map of vital sign measures.

In the method, it is possible that the at least one reflection image is divided into a number of partial reflection images and for each of the partial reflection images an individual vital sign measure is determined. When determining individual vital sign measures for the number of partial reflection images, it is possible to perform statistics with the individual vital sign measures such as averaging or weighing. For example, it is possible to provide a combined vital sign measure based on individual vital sign measures associated with the number of partial reflection images. A combined vital sign measure may be generated by assigning a larger weight to individual vital sign measures associated with partial reflection images that are located, e.g., in a central area of the reflection image and assigning a smaller weight to individual vital sign measures associated with partial reflection images that are located, e.g., closer to the border of the reflection image. Such a combined vital sign measure may have an improved reliability with regard to whether a living being has actually been presented to a camera.

Alternatively, in the method, it is possible that the at least one reflection image is divided into a number of partial reflection images and that for only some partial reflection images of the number of partial reflection images an individual vital sign measure is determined. For example, considering only some partial reflection images may comprise considering a reduced number of partial reflection images that is at least by one smaller than the total number of partial reflection images. It is possible, that only those partial reflection images are considered for determining an individual vital sign measure that are associated with a certain area of the respective reflection image. For example, the reflection image may be divided into quadrants or segments and only those partial reflection images are considered for determining an individual vital sign measure that lie within a certain quadrant or area, for example, in a top right quadrant or in a central area.

In this case, when dividing the at least one reflection image into a number of partial reflection images, it is possible to determine a respective individual vital sign measure only for those partial reflection images that lie within a specific quadrant or area of the reflection image. In case, several reflection images are used, it is possible, that the quadrants or areas are identical for all reflection images. However, it is also possible that the respective quadrants or areas of respective reflection images do not overlap or only partly overlap.

Considering only a fraction of partial reflection images, i.e., only some partial reflection images, for determining individual vital sign measures may have the advantage that more relevant or meaningful parts of a reflection image can be selected or extracted for determining individual vital sign measures. Thereby, the obtained individual vital sign measures can be more reliable. It is possible that several individual vital sign measures are combined to a combined vital sign measure, e.g., by averaging or weighing. The combined vital sign measure obtained from the selected individual vital sign measures may be more reliable in comparison to a single vital sign measure determined for the whole original reflection image from which the partial reflection images have been obtained.

It is also possible that for providing the map of vital sign measures, the reflection image may be divided into a number of partial reflection images. Preferably, for each of the partial reflection images or at least most of the partial reflection images, an associated speckle contrast is determined and used for determining an individual vital sign measure for each partial reflection image. It is further preferred that each of the partial reflection images at least partially overlaps with another partial reflection image of the number of partial reflection images. In particular, if the partial reflection images at least partially overlap, the series of partial reflection images may resemble a sliding view of a small section of the full reflection image.

Alternatively, a map can be obtained from a set of reflection images received from a camera used for recording the several reflection images, e.g., by scanning the object. The set of reflection images may be represented by the image data set. Preferably, each reflection image of the set of reflection images is associated with an individual scanning position, e.g., a position on the object or a position of the camera at the time of recording the respective image. The position associated with a reflection image of the set of reflection images may be defined in the coordinate system of the object or in the coordinate system of the camera.

In some variants of the method, the image data set represents at least two reflection images of speckle patterns produced by coherent electromagnetic radiation reflected from the object at different spatial positions on said object. Preferably, for each of the at least two reflection images, a vital sign measure associated with the respective spatial position is determined. The vital sign measure associated with the respective spatial position may be provided as a map of vital sign measures.

If the image data set represents at least two reflection images, it may be preferable, if motion correction is performed on a reflection image of the at least two reflection images. In particular, motion correction is beneficial if the object has moved while recording the at least two reflection images.

If at least two reflection images are used, e.g., for generating a map of vital sign measures, it may be beneficial that each or at least some of the at least two reflection images is divided into a number of partial reflection images as described before. Preferably, for each or at least most of the partial reflection images, an individual vital sign measure may be determined. The number of individual vital sign measures associated with respective partial reflection images may be provided as a map of vital sign measures. Thereby it is possible to increase the resolution of a map of vital sign measures without the need of recording even a larger number of reflection images.

Preferably, the vital sign measure is determined using an algorithm that may implement a mechanistic model or a data-driven model. The data-driven model may be a classification model such as a neural network that is trained for determining a vital sign measure, a vision transformer that is configured for determining a vital sign measure or the like. The mechanistic model, preferably, reflects physical phenomena in mathematical form, e.g., including first-principle models. A mechanistic model may comprise a set of differential equations that describe an interaction between the object and the coherent electromagnetic radiation thereby resulting in a specific speckle contrast. In particular, flow of a fluid and/or the geometry of the object may be represented by the mechanistic model. Based on the required input to the mechanistic model leading to a speckle contrast as determined from the speckle pattern of the at least one reflection image, an associated vital sign measure can be determined with the mechanistic model.

The thus determined vital sign measure may be indicative of a likelihood of the object being a living subject. As stated before, the possible speckle contrast values are generally distributed between 0 and 1, with 0 representing maximal blurring and thus a maximum likelihood in favour of a presence of a living subject and with 1 representing minimum or even no blurring and thus a maximum likelihood that no living subject is present. The determined vital sign measure may thus indicate based on the obtained speckle contrast value as obtained, e.g., with the mechanistic model, a certain likelihood that a living subject has been presented to the camera. For example, the vital sign measure may indicate a likelihood of 100% for a living subject being present if the determined speckle contrast is 0. However, the vital sign measure may indicate a likelihood of 0% for a living subject being present if the determined speckle contrast is 1. A speckle contrast of 0.5 may lead to a vital sign measure indicating a likelihood of 50% for a living subject being present. Of course, it may be possible that a speckle contrast does not need to translate in a one-to-one manner to a corresponding percentage represented by a vital sign measure. For example, a speckle contrast value of, e.g., 0.6 may lead to a vital sign measure indicating a likelihood of at least 75% that an object presented to a camera is a living subject.

Preferably, the data-driven model is a trained neural network configured for predicting the vital sign measure for the at least one reflection image based on the determined speckle contrast. For training the neural network, historic data may be used, representing speckle contrast values determined from a plurality of reflection images. Preferably, the neural network is trained to use a determined speckle contrast or a speckle contrast map as input and to output a vital sign measure or a vital sign measure map.

The trained neural network can be a multi-scale neural network or a recurrent neural network (RNN) such as, but not limited to, a gated recurrent unit (GRU) recurrent neural network or a long short-term memory (LSTM) recurrent neural network. Alternatively, the neural network may be a convolutional neural network (CNN).

The neural network may be trained using training data, e.g., comprising speckle contrast values and associated vital sign measures. For example, if the neural network is a feedforward neural network such as a CNN, a backpropagation-algorithm may be applied for training the neural network. In case of a RNN, a gradient descent algorithm or a backpropagation-through-time algorithm may be employed for training purposes. As a result of the training, operating parameters for the neural network circuitry are generated such that when receiving a speckle contrast as input, the trained neural network outputs an associated vital sign measure as a prediction.

In some variants of the method, the image data set represents at least two reflection images of speckle patterns produced by coherent electromagnetic radiation reflected from the object at the same or at least similar spatial position in the object. Reflection images recorded at similar spatial positions, preferably, represent a significant spatial overlap. Preferably, for each of the images, a vital sign measure is determined and provided based on the speckle contrast of the respective reflection image. Thereby, a series of reflection images may be produced representing an evolution of a speckle pattern as a function of time. From each of the speckle patterns, a speckle contrast and an associated vital sign measure may be determined. Accordingly, it may also be possible to provide an evolution of, e.g., a vital sign measure as a function of time. Considering blurring of the speckle pattern induced by blood flow, by recording and processing a series of reflection images representing speckle patterns of the same spatial position on an object, variations of the blood amount in the illuminated volume, e.g., caused the heart activity, may be obtained.

The vital sign measure may be used for detecting a living subject, such as a human or an animal. For example, the method for detecting a vital sign may be used as part of an authentication process implemented on a device for providing access control for a user trying to access the device. A device may be, for example, a cell phone or a tablet computer or a smart watch.

Preferably, the method further includes the step of predicting a presence of a living subject based on the provided vital sign measure, preferably, as part of an authentication process.

Preferably, the step of predicting of the presence of a living subject based on the provided vital sign measure includes at least one of the sub-steps of
determining a confidence score based on the determined vital sign measure,
comparing the confidence score to a predefined confidence threshold, and
predicting the presence of a living subject based on the comparison.

The confidence score may be generated from a vital sign measure, e.g., represented by a single number or a value, or from a vital sign map, e.g., represented by a matrix of vital sign measures. The confidence score may represent a degree of confidence indicative of a presence of a living subject. The confidence score may be expressed by a single number or a value.

Preferably, the confidence score is determined by comparing the determined vital sign measure to a reference, e.g., to one or more reference vital sign measures each being preferably associated with a particular confidence score.

Alternatively, the confidence score may be determined using a neural network that is trained for receiving the determined vital sign measure as input and for providing the confidence score as output. The neural network may be trained with historic data representing historic vital sign measures and associated confidence scores.

The confidence threshold is predetermined to ensure a certain level of confidence that the object indeed is a living subject. The confidence threshold may be predetermined in dependence on a security level required for a specific application, e.g., for providing access to a device. For example, the confidence threshold may be set such that the confidence score may represent a comparatively high level of confidence, e.g., 90% or more, e.g., 99% or more, that the object presented to a camera is a living subject. Only when the comparison with the confidence threshold yields that the confidence score is high enough, i.e., exceeding the confidence threshold, the presence of a living subject is approved. If the confidence score is below the confidence threshold, access to the device will be denied. A denial of access may trigger a new measurement, e.g. a repetition of the method of detecting a vital sign and of making use of the vital sign measure for predicting the presence of a living subject as described before. Optionally, an alternative authentication process may be Thereby, it is possible to make sure that a requestor trying to gain access to a device actually is a living subject and not a spoofing attach. Additionally, it is possible to make sure that a requestor is authorized for the particular request. Accordingly, in the method, it is particularly preferred that it comprises a step of authenticating an object, e.g., a user, based on the determined confidence score.

In the method, it is also possible to determine a combined confidence score by combining at least two determined individual confidence scores. For example, in the method it is possible that from at least two reflection images, a combined confidence score is determined based on at least two confidence scores determined for the at least two reflection images, respectively. Obtaining a combined confidence score may be achieved, e.g., by averaging or weighting at least two confidence scores determined for the at least two reflection images. When determining a combined confidence score, it is possible to achieve an even higher level of confidence that an object indeed is in fact a living subject. This may be particularly useful as part of an authentication process.

The above-described method for determining a vital sign of an object and the above-described method for predicting a presence of a living subject using a determined vital sign may be part of an authentication process, in particular, further including biometric authentication, e.g., facial recognition and/or fingering sensing.

An authentication process may comprise the following steps:
performing biometric recognition of a user, e.g., on a user's face presented to a camera, or by determining a user's fingerprint with a fingerprint sensor, preferably, by conducting the sub-steps of
   providing a detector signal from a camera, said detector signal representing an image of a user's feature, e.g., a fingerprint feature or a facial feature;
   generating a low-level representation of the image; and
   validating an authorisation of the user based on the low-level representation of the image and a stored low-level representation template,
if the biometric recognition is successful, determining a vital sign of the user, preferably, by conducting the steps of the method for determining a vital sign of an object as described before,
predicting based on the determined vital sign a presence of a living subject, preferably, by
   determining a confidence score based on a determined vital sign measure,
   comparing the confidence score to a predefined confidence threshold, and
   predicting the presence of a living subject based on the comparison;
providing a positive authentication output signal if the presence of a living subject is verified.

Upon receiving a positive authentication output signal the user may be allowed to access the device. Otherwise, in case of no living subject could be detected, a negative authentication output signal may be provided. In other words, generally, an authentication output signal may be provided indicative of whether a living subject has been presented to the camera. In case the biometric authentication already yields a negative result, a negative authentication output signal may be provided without determining the vital sign of the object.

In an alternative authentication process, initially a vital sign of the object is determined and afterwards, in case of a successful verification of a presence of a living subject, the biometric authentication, e.g., facial recognition or fingerprint sensing, is carried out.

The invention also relates to a computer program for detecting a vital sign of an object, the computer program including instructions for executing the steps of the method for determining a vital sign of an object as described before, when run on a computer.

The invention also relates to a non-transitory computer readable data medium storing a computer program including instructions for executing the steps of the method for determining a vital sign of an object as described before.

With regard to the system, the above objective is achieved by a system for detecting a vital sign. The system comprises a generating or receiving unit, a processor, and a providing unit.

The generating or receiving unit is configured for generating or receiving an image data set representing at least one reflection image of a speckle pattern produced by coherent electromagnetic radiation reflected from an object. The processor is configured for determining a speckle contrast of the speckle pattern, and for determining a vital sign measure based on the determined speckle contrast. The providing unit is configured for providing the vital sign measure.

The system, preferably, is configured for carrying out the method of detecting a vital sign as described before. For example, the system may comprise the computer program for detecting a vital sign of an object, the computer program including instructions for executing the steps of the method for determining a vital sign of an object as described before, when run on a computer. The system may comprise the non-transitory computer readable data medium storing this computer program for determining a vital sign of an object.

The generating or receiving unit may be implemented as an input configured for receiving image data or for generating image data from a received image signal. The processor may be an image signal processor (ISP) and may include circuitry suitable for processing reflection images, in particular, reflection images received from the generating or receiving unit.

For determining a vital sign measure based on the determined speckle contrast, the processor may include a neural network module comprising a neural network configured for using a speckle contrast as input and for outputting an associated vital sign measure.

The providing unit may be configured as an output for providing the vital sign measure, e.g., in form of vital sign measure data.

It shall be understood that the aspects described above, and specifically the method of claim 1, the system of claim 16 and the computer program of claim 14, have similar and/or identical preferred embodiments, in particular as defined in the dependent claims.

It shall be understood that a preferred embodiment of the invention can also be any combination of the dependent claims or above embodiments with the respective independent claim.

These and other aspects of the invention will be apparent from and elucidated with reference to the embodiments described hereinafter.

### BRIEF DESCRIPTION OF THE DRAWINGS

FIG. 1 shows a flowchart representing a method for detecting a vital sign;

FIG. 2 shows a flowchart representing a use of a vital sign measure for predicting a presence of a living subject, in particular, as part of an authentication process;

FIG. 3 shows a schematic representation of a system for detecting a vital sign;

FIG. 4 shows a vital sign measure map of vital signs of parts of a human hand; and

FIG. 5 shows a flowchart representing an authentication process comprising the determination of a vital sign.

### DETAILED DESCRIPTION OF EMBODIMENTS

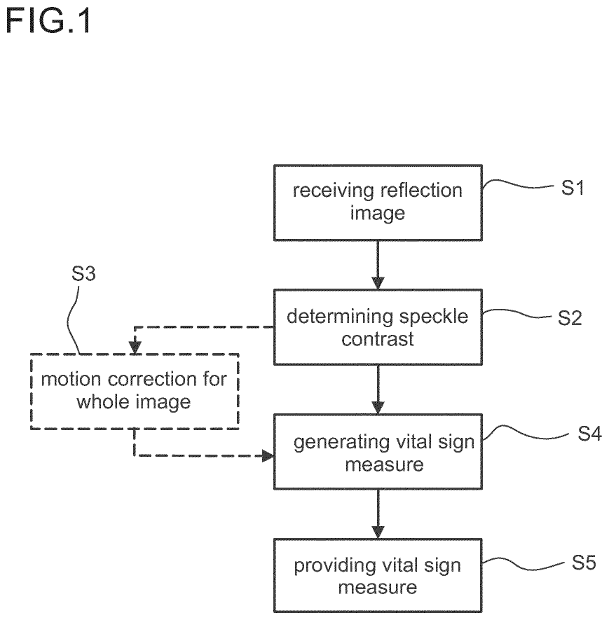

FIG. 1 shows a flowchart representing a method for detecting a vital sign. In the method, a reflection image represented by an image data set is received (step S1). The reflection image shows a speckle pattern. The speckle pattern was produced by illuminating an object with coherent electromagnetic radiation having a line width having a centre wavelength that is between 500 nm and 560 nm, i.e., green light. Green light is expected to provide a comparatively high contrast in a speckle pattern due to the interaction with haemoglobin in blood. Alternatively, it is preferred that infrared light is used. Infrared light has the advantage that is not visible for human beings and thus causes less nuisance to a user. The coherent electromagnetic radiation is reflected from the object and thereby a speckle pattern is generated due to random interference. The reflected light is captured with a camera for recording the speckle pattern.

Alternatively, more than one reflection image, e.g., at least two reflection images, represented by the image data set may be received. Each of the reflection images, preferably, represents a speckle pattern obtained by illuminating the object with coherent electromagnetic radiation and by recording the diffuse reflection from the object's surface with a camera. The at least two images may be recorded at the same or at least at a similar spatial position on the object or at different spatial positions, i.e. with no or only a small overlap, on the object, e.g., by scanning the object.

From the reflection image, a speckle contrast is determined (step S2). In case, at least two reflection images are received, for each of the reflection images preferably a speckle contrast is determined. The several speckle contrasts can be combined in a speckle contrast map, e.g., represented by a matrix.

The speckle contrast is determined by the standard deviation of the illumination divided by the mean intensity. The speckle contrast may range between 0 and 1 whereas the speckle contrast is 1 in case of no blurring of the speckles, i.e., no motion was detected in the illuminated volume of the object, and the speckle contrast is 0 in case of maximum blurring of the speckle due to detected motion of particles, e.g., red blood cells, in the illuminated volume of the object. Thus, the speckle pattern and the speckle contrast derived therefrom are sensitive to motion in the illuminated volume. This motion in the illuminated volume is indicative of the object being a living subject, since a living subject like a human or an animal has circulatory system for transporting blood cells within the body. In case, no blood circulation can be detected, it can be expected that an object is not a living subject.

The stronger the blurring of the speckles is, the smaller is the derived speckle contrast. Thus, with a decreasing speckle contrast it is possible to more reliably detect a vital sign of an object.

The speckle contrast can be determined from the full reflection image or from a section of the reflection image, e.g., obtained by cropping the full reflection image.

Optionally, motion correction can be performed on the reflection image (step S3), in case the object has moved while capturing the reflection image.

From the speckle contrast, a vital sign measure is determined (step S4). If a speckle contrast map has been determined from a series of reflection images, a vital sign measure map can be generated. A vital sign measure map may also be generated from a single reflection image, by dividing the reflection image into a number of partial reflection images and by determining a speckle contrast for each of the partial images. Based on each of the speckle contrasts, implementing a speckle contrast map, a corresponding vital sign measure may be determined. The thus determined vital sign measures can be combined to a vital sign measure map. Thereby, the vital sign measures can be more accurately matched to certain positions on the object. In other words, it is possible to find a contribution of a part of the object to the total vital sign measure. For example, parts of an object exhibiting a comparatively high motion of fluid are expected to contribute more prominently to a total vital sign measure associated with the complete volume illuminated by coherent electromagnetic radiation.

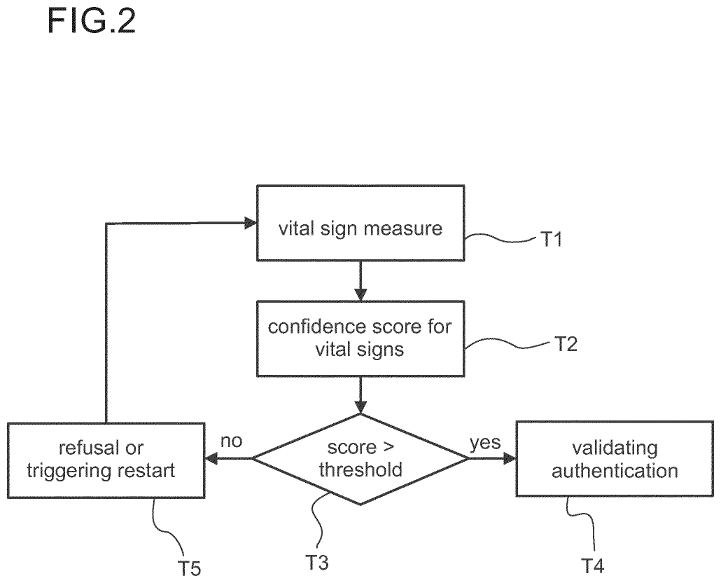

Afterwards, the determined vital sign measure or the vita sign measure map is provided (step S5), e.g., to a user or to another component of the same device or to another device or system for further processing. For example, the vital sign measure may be used for predicting a presence of a living subject, e.g., as part of an authentication process implemented in a device as described with reference to FIG. 2. In particular, the vital sign measure is indicative of an object exhibiting a vital sign. The vital sign measure can thus be used for assessing whether an object presented to the camera is a living subject.

FIG. 2 shows a flowchart representing a use of a vital sign measure for predicting a presence of a living subject, in particular, as part of an authentication process.

Initially, a vital sign measure or a vital sign measure map is provided (step T1). The vital sign measure, e.g., a single number, or the vital sign measure map, e.g., a matrix, can be determined and provided by executing the method for detecting a vital sign as described with reference to FIG. 1.

Based on the vital sign measure or the vital sign measure map, a confidence score is determined (step T2). For generating the confidence score, a trained neural network is used. The neural network is trained to receive a vital sign measure or a vital sign measure map and to output a confidence score that is based on the vital sign measure or the vital sign measure map, respectively. For example, the neural network can be trained using training data representing pairs of a vital sign measure or a vital sign measure map and a corresponding confidence score.

Alternatively, the confidence score may be generated by comparing the vital sign measure or the vital sign measure map to a reference. Depending on the degree of matching of the vital sign measure or the vital sign measure map with the reference the confidence score may be generated. For example, a number of intervals may be defined around the reference and depending on the respective interval, in which the vital sign measure or the vital sign measure map lies, the confidence score is generated.

The confidence score indicates based on the vital sign measure or the vital sign measure map a degree of confidence, that the object presented to a camera actually is a living subject. For example, if a vital sign measure has a certain number derived from the related speckle contrast, based on this vital sign measure the confidence score is generated. If the vital sign measure indicates with that most certainly the object is a living subject, the confidence score generated therefrom may indicate a confidence of, e.g., 95% or more that the object indeed is a living subject.

The generated confidence score is subsequently compared to a predefined confidence threshold (T3). The confidence threshold is predefined according to a security level required for a specific application. If the security level is high, e.g., in case of conducting online payment or the like, the confidence threshold may be set to only allow a confidence score indicative of an almost 100% certainty of the object presented to the camera is a living subject may be required to exceed the confidence threshold. In case, access to a less sensitive application is requested, the confidence threshold may be predefined to require a comparatively lower confidence score to allow access to this particular application.

If the confidence score exceeds the confidence threshold, the authentication may be validated (step T4). Otherwise, the request may be refused or a new measurement may be triggered (Step T5).

The method described with reference to FIG. 2 can be combined with other factors, like facial recognition or a finger print scan. Thereby, it is possible to make sure that a requestor is really a living subject and not a spoofing attach, and, additionally, it can be made sure that the requestor is actually authorized for the particular request. As a result, the security level can be further enhanced.

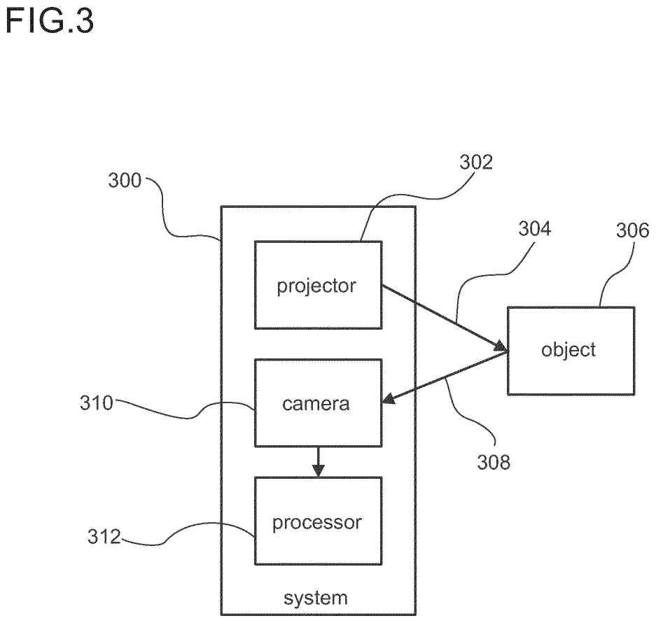

FIG. 3 shows a schematic representation of a system 300 for detecting a vital sign. The system 300 comprises a projector 302 that is configured for projecting coherent electromagnetic radiation along a transmit path 304 to an object 306 (object 306 is not part of system 300). The projector 302 can be laser. The object 306 can be a body part of a human such the human's face or hand. The projector 302 can be selected from a group of projectors emitting coherent electromagnetic radiation at different wavelengths. Preferably, projector 302 is configured to emit coherent electromagnetic radiation that has a line width with a centre wavelength that is between 500 nm and 560 nm, i.e. green light, or that is between 780 nm and 3000 nm, i.e. lying in the infrared (IR) spectrum, preferably, between 780 nm and 1000 nm, i.e., lying in the near infrared spectral range. Green light usually provides the highest contrast due to the interaction with the hemoglobin in the blood. IR light has the advantage that it is invisible and thus causes less nuisance to the user.

The object 306 reflects the coherent electromagnetic radiation along a return path 308 and the reflected electromagnetic radiation is captured by a camera 310. By reflecting the coherent electromagnetic radiation from the object 306, a speckle pattern is produced in which a spatial intensity randomly varies due to interference of coherent wave fronts. The camera 310 thus records a reflection image of a speckle pattern produced by coherent electromagnetic radiation reflected from an object. The speckle pattern is sensitive to motion in the illuminated volume, e.g., of red blood cells transported in a circulatory system of the object. In particular, case of moving particles in the illuminated volume, the speckles become blurred such that a speckle contrast derived from the speckle pattern decreases. The degree of blurring of speckle pattern is an indicator for the presence of a living subject having a circulatory system.

In particular, generally, an outer surface of the object 306 causes a specular reflection and an inner part of the sample causes a diffuse reflection. Usually, the specular reflection overpowers the diffuse reflection. Since a speckle pattern typically occurs in diffuse reflections of coherent electromagnetic radiation, it can be advantageous to use a polarization filter to eliminate most of the specular reflection.

The camera 310 records the reflection image and passes it in form of an image data set or in form of an image signal to a processor 312. For recoding the reflection image, the camera 310, preferably, includes one or more lenses and one or more image sensors. An image sensor may be, for example, an array of sensors. Sensors in the sensor array may include, but are not be limited to, charge coupled device (CCD) or complementary metal oxide semiconductor (CMOS) sensor elements to capture the reflected coherent electromagnetic radiation.

The processor 312, preferably, is configured for executing the method steps as described with reference to FIG. 1. In particular, the processor 312 may comprise a generating or receiving unit configured for generating or receiving an image data set representing a reflection image of a speckle pattern produced by coherent electromagnetic radiation reflected from the object 306. For example, the generating or receiving unit may be configured as an input of processor 312 for receiving image data from the camera 310 representing the captured reflection image. Alternatively, the input may be configured for receiving an image signal from the camera 310. Based on the image signal, the image data set can be generated for further processing by the processor 312. Preferably, the processor 312 comprises a logical circuit for processing the image signal and/or the image data set provided by the camera 310.

The processor 312 is configured for determining a speckle contrast of the speckle pattern shown in the reflection image. For determining the speckle contrast of the speckle pattern, the processor 312 is configured to calculate the standard deviation of the illumination divided by the mean intensity. A resulting speckle contrast generally lies in the range between 0 and 1. A speckle contrast of 1 indicates no blurring of the speckles, i.e., no motion in the illuminated volume of the object 306, and a speckle contrast of 0 indicates maximum blurring of the speckles due to detected motion of particles, e.g., red blood cells, in the illuminated volume of the object 306.

The processor 312 is further configured for determining a vital sign measure based on the determined speckle contrast. The processor 312 is also configured for determining a vital sign measure map based on a determined speckle contrast map as described before.

To this end, the processor 312 has a neural network module comprising trained neural network. The neural network is trained for predicting the vital sign measure for the reflection image based on the determined speckle contrast. Accordingly, the neural network is trained to use a speckle contrast determined by the processor as input and to provide a vital sign measure as output. The trained neural network may be, for example, a multi-scale neural network or a recurrent neural network (RNN) such as a gated recurrent unit (GRU) recurrent neural network or a long short-term memory (LSTM) recurrent neural network. Alternatively, the neural network may be a convolutional neural network (CNN).

Alternatively or additionally to a trained neural network, the processor 312 may comprise an algorithm that may implement a mechanistic model. The mechanistic model is configured for determining the vital sign measure using based on first-principle assumptions.

The processor 312 may further comprise a providing unit for providing the determined vital sign measure. The providing unit may be an output of processor 312 and configured for providing output data or an output signal representing the determined vital sign measure. A vital sign measure determined by system 300 can be used for predicting a presence of a living subject, e.g., by executing the method steps as described with reference to FIG. 3, preferably, as part of an authentication process.

It is possible that system 300 only comprises processor 312 and not projector 302 and camera 310. In this case, system 300 and, in particular, processor 312 is operatively connected to the projector 302 and/or camera 310 for exchanging one or more reflection images.

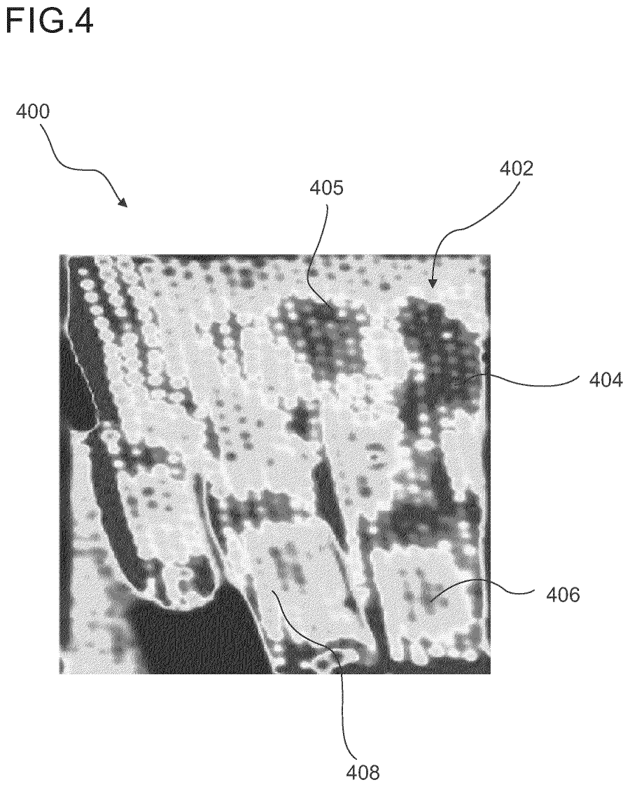

FIG. 4 shows a vital sign measure map 400 of vital signs of parts of a human hand 402. The vital signs map can be generated by conducting the method as described with reference to FIG. 1 and/or by using the system 300 as described with reference to FIG. 3.

The vital sign measure map 400 comprises a plurality of vital sign measure values that are visualized according to their relative position on the illuminated part of the hand 402. In other words, each of the vital sign measure values has an associated coordinate in the coordinate system of the hand 402 and the map is visualized in the coordinate system of the hand 402. The vital sign measure values thus represent a vital sign measure determined from a speckle pattern produced by illuminating the respective position on the hand 402 by means of coherent electromagnetic radiation.

In the map 400, the individual visual sign measures are visualized in a colour coding representing a degree of blood flow in the respective part of the hand 402. Thus, from the individual vital sign measures areas 404, 405 of the hand 402 can be determined showing a high amount of blood flow. Accordingly, other areas 406 and 408 can be determined showing a comparatively low amount of blood flow.

It is in particular an area 404, 405 of the hand 402 showing a high amount of blood flow that may result in a vital sign measure based on which a confidence score may be determined indicating with comparatively high confidence a presence of a living subject. In contrast, areas 406 and 408 representing a comparatively low amount of blood flow may lead to a confidence value indicating with comparatively lower confidence the presence of a living subject. In an authentication process, a confidence score that was generated based on a vital sign measure of area 404 or area 405 may thus lead to a validation of the authentication whereas a confidence value based on a vital sign measure of area 406 or 408 may lead to a refusal of the authentication. Alternatively, a restart of the authentication process may be triggered, e.g., this time selecting a different position on the hand for generating the confidence score.

The vital sign measure map 400 may be generated from a single reflection image, e.g., by dividing the reflection image into a plurality of partial reflection images. For each of the partial reflection images a vital sign measure can be determined and combined to the vital sign measure map 400.

Alternatively, vital sign measure map 400 may be generated from a number of reflection images that have been recorded by scanning over the hand 402. For each of the number of reflection images, it is preferred that position information being included for combining the vital sign measures of the individual reflection images to vital sign measure map 400. To increase to resolution of the vital sign measure map, each of the number of reflection images by be further divided into a number of partial images and for each of the partial images an individual vital sign measure may be determined.

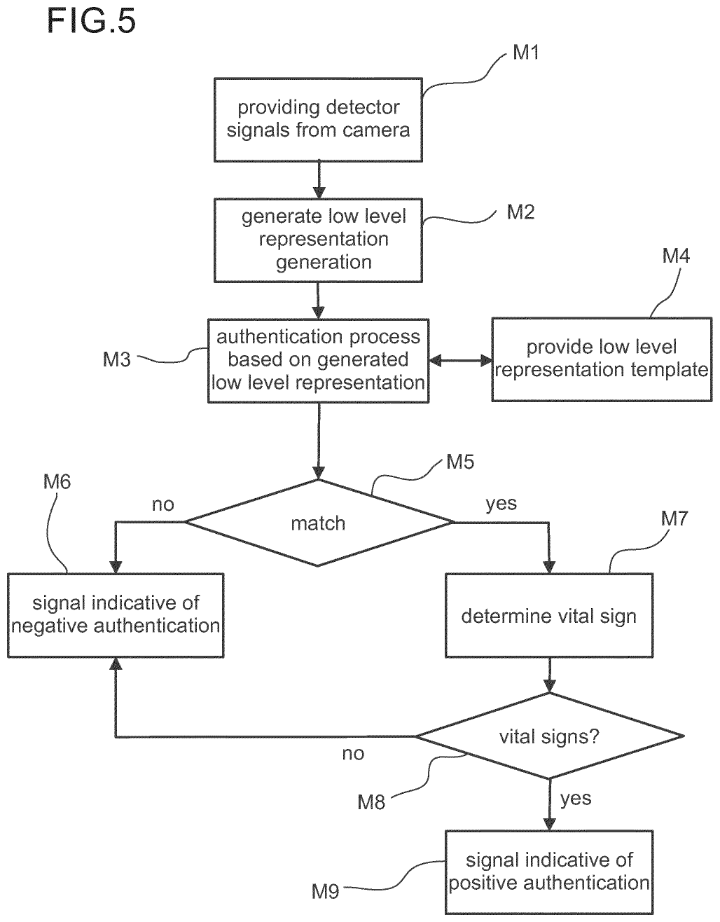

FIG. 5 shows a flowchart representing an authentication process comprising the determination of a vital sign.

Initially, a detector signal from a camera is provided that, e.g., represents an image of a fingerprint captured with a fingerprint sensor or an image of a face, e.g., in case of facial recognition (step M1). The detector signal may be provided upon receiving an unlock request for the device from a user. The unlock request may trigger, e.g., in case of facial recognition, an illumination of the user's face with flood infrared illumination and patterned infrared illumination. The reflected light may be captured by the camera to provide a detector signal representing an image of the illuminated body part, e.g., a face.

Of the captured image, a low-level representation is generated (step M2). A low-level representation may be generated by employing, e.g., fast Fourier transform (FFT), wavelets, deep learning, like a CNN, energy models, normalizing flows, vision transformers, or autoregressive image modelling.

The biometric authentication is performed based on the generated low-level representation (step M3). To this end, a low-level representation template is provided (step M4). For example, analysed facial or fingerprint features may be compared to the corresponding template. The template may be provided to get a matching score. In certain embodiments, a template space may include a template for an enrolment profile for an authorized user on device, e.g., a template generated during an enrolment process. A matching score may be a score of the differences between facial or fingerprint features and corresponding features in template space, e.g., feature vectors for the authorized user generated during the enrolment process. A matching score may be higher when feature vectors are closer to, e.g., the less distance or less difference, the feature vectors in template space.

Comparing feature vectors and templates from a template space to get a corresponding matching score may include using one or more classifiers or a classification-enabled network to classify and evaluate the differences between the generated feature vectors and feature vectors from the template space. Examples of different classifiers that may be used include, but are not limited to, linear, piecewise linear, nonlinear classifiers, support vector machines, and neural network classifiers. In some embodiments, matching score may be assessed using distance scores between feature vectors and templates from the template space.

For authentication, a matching score may be compared to an unlock threshold for device (step M5). The unlock threshold may represent a minimum difference in feature vectors, e.g., between the face of the authorized user according to templates and the face of the user in the unlock attempt to unlock the device. For example, an unlock threshold may be a threshold value that determines whether the unlock feature vectors are close enough to the template vectors associated with the authorized user's face.

If the matching score is below the unlock threshold, the user's face or fingerprint in the captured image for unlocking does not match the face or fingerprint of the authorized user. In this case, a signal indicative of negative authentication is provided (step M6).

Yet, if the matching score is above unlock threshold, the user's face or fingerprint in the captured image for unlocking matches the face or fingerprint of the authorized user. In this case, a second authentication process may be initiated.

This second authentication process comprises a determination of a vital sign of the user (step M7). For determining the vital sign, the method for determining a vital sign as described with reference to FIG. 1 may be used. For example, the method described with reference to FIG. 1 may be carried out using the system as described with reference to FIG. 3.

Based on the determined vital sign it is verified whether the determined vital sign indicates the presence of a living subject (step M8), e.g., by executing the method for predicting a presence of a living subject as described with reference to FIG. 2. For example, when combined with a facial recognition process or fingerprint recognition, it may be determined whether the part of the user presented to the camera, e.g., face or finger shows a vital sign being indicative of the presence of a living subject, and not, e.g. a spoofing attach imitating the respective body part of the authorized user.

If the presence of a living subject is denied, a signal indicative of a negative authentication may be provided (corresponding to step M6).

However, if the user also successfully passes the second authentication step, i.e., the presence of a living subject is verified, a signal indicative of positive authentication may be provided (step M9).

As a result, the user is verified as the authorized user for the enrolment profile on the device and the device is unlocked. Unlocking may allow the user to access or use the device and/or allowing the user to have access to a selected functionality of the device, e.g., unlocking a function of an application running on the device, payment systems or making a payment, access to personal data, expanded view of notifications, etc.

Alternatively, the authentication process may be carried out in way that initially a vital sign is determined and used for predicting a presence of a living subject and only if the presence of a living subject is verified, afterwards, another second authentication process is carried out such as biometric authentication including, e.g., facial recognition or fingerprint sensing.

Other variations to the disclosed embodiments can be understood and effected by those skilled in the art in practicing the claimed invention, from a study of the drawings, the disclosure, and the appended claims.

The term “reflection image” as used herein is not limited to an actual visual representation of the imaged object. Instead, an “image” as referred to herein can be generally understood as a representation of the imaged object in terms of image data acquired by imaging the object, wherein “imaging” can refer to any process involving an interaction of electromagnetic waves, particularly light or radiation, with the object, specifically by reflection, for instance, and a subsequent capturing of the electromagnetic waves using an optical sensor, which might then also be regarded as an image sensor. In particular, the term “reflection image” as used herein can refer to image data based on which an actual visual representation of the imaged object can be constructed. For instance, the image data can correspond to an assignment of color or grayscale values to image positions, wherein each image position can correspond to a position in or on the imaged object. The images or image data referred to herein can be two-dimensional, three-dimensional or four-dimensional, for instance, wherein a four-dimensional image is understood as a three-dimensional image evolving over time and, likewise, a two-dimensional image evolving over time might be regarded as a three-dimensional image. A reflection image can be considered a digital image if the image data are digital image data, wherein then the image positions may correspond to pixels or voxels of the image and/or image sensor.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

Procedures like generating or receiving an image data set representing at least one reflection image of a speckle pattern, determining a speckle contrast of the speckle pattern, determining a vital sign measure based on the determined speckle contrast, and providing the vital sign measure, etc. performed by one or several units or devices can be performed by any other number of units or devices. These procedures can be implemented as program code means of a computer program and/or as dedicated hardware.

A computer program may be stored/distributed on a suitable medium, such as an optical storage medium or a solid-state medium, supplied together with or as part of other hardware, but may also be distributed in other forms, such as via the Internet or other wired or wireless telecommunication systems.

Any units described herein may be processing units that are part of a classical computing system. Processing units may include a general-purpose processor and may also include a field programmable gate array (FPGA), an application specific integrated circuit (ASIC), or any other specialized circuit. Any memory may be a physical system memory, which may be volatile, non-volatile, or some combination of the two. The term “memory” may include any computer-readable storage media such as a non-volatile mass storage. If the computing system is distributed, the processing and/or memory capability may be distributed as well. The computing system may include multiple structures as “executable components”. The term “executable component” is a structure well understood in the field of computing as being a structure that can be software, hardware, or a combination thereof. For instance, when implemented in software, one of ordinary skill in the art would understand that the structure of an executable component may include software objects, routines, methods, and so forth, that may be executed on the computing system. This may include both an executable component in the heap of a computing system, or on computer-readable storage media. The structure of the executable component may exist on a computer-readable medium such that, when interpreted by one or more processors of a computing system, e.g., by a processor thread, the computing system is caused to perform a function. Such structure may be computer readable directly by the processors, for instance, as is the case if the executable component were binary, or it may be structured to be interpretable and/or compiled, for instance, whether in a single stage or in multiple stages, so as to generate such binary that is directly interpretable by the processors. In other instances, structures may be hard coded or hard wired logic gates, that are implemented exclusively or near-exclusively in hardware, such as within a field programmable gate array (FPGA), an application specific integrated circuit (ASIC), or any other specialized circuit. Accordingly, the term “executable component” is a term for a structure that is well understood by those of ordinary skill in the art of computing, whether implemented in software, hardware, or a combination. Any embodiments herein are described with reference to acts that are performed by one or more processing units of the computing system. If such acts are implemented in software, one or more processors direct the operation of the computing system in response to having executed computer-executable instructions that constitute an executable component. Computing system may also contain communication channels that allow the computing system to communicate with other computing systems over, for example, network. A “network” is defined as one or more data links that enable the transport of electronic data between computing systems and/or modules and/or other electronic devices. When information is transferred or provided over a network or another communications connection, for example, either hardwired, wireless, or a combination of hardwired or wireless, to a computing system, the computing system properly views the connection as a transmission medium. Transmission media can include a network and/or data links which can be used to carry desired program code means in the form of computer-executable instructions or data structures and which can be accessed by a general-purpose or special-purpose computing system or combinations. While not all computing systems require a user interface, in some embodiments, the computing system includes a user interface system for use in interfacing with a user. User interfaces act as input or output mechanism to users for instance via displays.

Those skilled in the art will appreciate that at least parts of the invention may be practiced in network computing environments with many types of computing system configurations, including, personal computers, desktop computers, laptop computers, message processors, hand-held devices, multi-processor systems, microprocessor-based or programmable consumer electronics, network PCs, minicomputers, main-frame computers, mobile telephones, PDAs, pagers, routers, switches, datacenters, wearables, such as glasses, and the like. The invention may also be practiced in distributed system environments where local and remote computing system, which are linked, for example, either by hardwired data links, wireless data links, or by a combination of hardwired and wireless data links, through a network, both perform tasks. In a distributed system environment, program modules may be located in both local and remote memory storage devices.

Those skilled in the art will also appreciate that at least parts of the invention may be practiced in a cloud computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources, e.g., networks, servers, storage, applications, and services. The definition of “cloud computing” is not limited to any of the other numerous advantages that can be obtained from such a model when deployed. The computing systems of the figures include various components or functional blocks that may implement the various embodiments disclosed herein as explained. The various components or functional blocks may be implemented on a local computing system or may be implemented on a distributed computing system that includes elements resident in the cloud or that implement aspects of cloud computing. The various components or functional blocks may be implemented as software, hardware, or a combination of software and hardware. The computing systems shown in the figures may include more or less than the components illustrated in the figures and some of the components may be combined as circumstances warrant.

Any reference signs in the claims should not be construed as limiting the scope.

1. A method for detecting a vital sign, the method comprising
generating or receiving an image data set (S1) representing at least one reflection image of a speckle pattern produced by coherent electromagnetic radiation reflected from an object,
determining a speckle contrast of the speckle pattern (S2),
determining a vital sign measure based on the determined speckle contrast (S4), and
providing the vital sign measure (S5).

2. The method of claim 1, comprising authenticating an object based on the provided vital sign measure.

3. The method of claim 2, wherein authenticating an object includes matching at least one biometric feature obtained from the object with at least one reference biometric feature associated with an authorised object.

4. The method of claim 1, wherein at least two vital sign measures, each being associated with a different spatial position on the object are determined and provided as a map of vital sign measures.

5. The method of claim 4, wherein for providing the map, the reflection image is divided into a number of partial reflection images and for each of the partial images an associated speckle contrast is determined and used for determining an individual vital sign measure for each partial reflection image.

6. The method of claim 4, wherein the image data set represents at least two reflection images of a speckle pattern produced by coherent electromagnetic radiation reflected from the object at different spatial positions on said object and wherein for each of the at least two reflection images, a vital sign measure associated with the respective spatial position is determined and provided as a map of vital sign measures.

7. The method of claim 6, wherein each of the at least two reflection images is divided into a number of partial reflection images, and for each of the partial reflection images an individual vital sign measure is determined.

8. The method of claim 1, wherein the vital sign measure is determined using a mechanistic model or a data-driven model.

9. The method of claim 1, wherein the image data set represents at least two reflection images of speckle patterns produced by coherent electromagnetic radiation reflected from the object at the same or at least overlapping spatial positions on the object and wherein for each of the reflection images a vital sign measure is determined and provided based on the speckle contrast of the respective reflection image.

10. The method of claim 1, further comprising a step of predicting a presence of a living subject based on the provided vital sign measure.

11. The method of claim 10, wherein the confidence score is determined by comparing the determined vital sign measure to a reference or by using a trained neural network.

12. The method of claim 10, comprising authenticating an object based on the determined confidence score.

13. The method of claim 10, wherein from at least two reflection images of speckle patterns, a combined confidence score is determined based on at least two confidence scores determined for the at least two reflection images, respectively.

14. A computer program for detecting a vital sign of an object, the computer program comprising instructions for executing the steps of the method according to claim 1, when run on a computer.

15. A non-transitory computer readable data medium storing the computer program of claim 14.

16. A system for detecting a vital sign, the system comprising
a generating or receiving unit configured for generating or receiving an image data set representing at least one reflection image of a speckle pattern produced by coherent electromagnetic radiation reflected from an object,
a processor configured for determining a speckle contrast of the speckle pattern, and for determining a vital sign measure based on the determined speckle contrast, and
a providing unit configured for providing the vital sign measure.

17. The method of claim 10, wherein the step of predicting a presence of a living subject based on the provided vital sign measure comprising at least one of the following sub-steps of
determining a confidence score based on the determined vital sign measure (T2),
comparing the confidence score to a predefined confidence threshold (T3), and
predicting the presence of a living subject based on the comparison (T4, T5).
